(19) United States
(12) Patent Application Publication
(10) Pub. No.: US 2006/0241543 A1
(43) Pub. Date: Oct. 26, 2006

(54) METHOD FOR INSTALLING AND SERVICING A WEARABLE CONTINUOUS RENAL REPLACEMENT THERAPY DEVICE

(75) Inventor: Victor Gura, Beverly Hills, CA (US)

Correspondence Address: HOWISON & ARNOTT, L.L.P, P.O. BOX 741715, DALLAS, TX, 75374-1715, US

(73) Assignee: NATIONAL QUALITY CARE, INC., Beverly Hills, CA (US)

(21) Appl. No.: 11/427,267

(22) Filed: Jun. 28, 2006

Related U.S. Application Data
(63) Continuation-in-part of application No. 10/940,862, filed on Sep. 14, 2004.
Application No. 10/940,862 is a continuation-in-part of application No. 10/085,349, filed on Nov. 16, 2001, now Pat. No. 6,960,179.

(51) Int. Cl. A61M 37/00

(52) U.S. Cl. 604/5.01

(57) ABSTRACT

A continuous renal replacement therapy (CRRT) device that is completely worn on the body of a patient. A protocol is provided for installing the completely wearable CRRT device on a patient and changing or exchanging various CRRT device parts and fluids at predetermined intervals. A dialyzer or hemofilter is changed in a sterile environment while the dialysate and dialysate filters can be changed in a non-sterile environment by the patient.

### CROSS REFERENCE TO RELATED APPLICATIONS

This application is a Continuation-In-Part application of U.S. Utility patent application Ser. No. 10/940,862, filed on Sep. 14, 2004, which is a Continuation-In-Part of Utility patent application Ser. No. 10/085,349, filed Nov. 16, 2001, which are incorporated herein by reference in their entirety.

### FIELD OF THE INVENTION

The present invention is directed to dialysis systems, and more particularly to a method of installing and servicing a dialysis system that may be continuously worn by a patient.

### BACKGROUND OF THE INVENTION

Hemodialysis is a process by which microscopic toxins are removed from the blood using a filtering membrane such as a dialyzer. Typically, hemodialysis is administered in intermittent three to four hours sessions, which take place two or three times per week. However, there exists a growing body of research that prefers daily dialysis since increased dialysis time improves outcomes both in terms of quality of life and longevity. However, the implementation of daily dialysis is almost impossible due to manpower and cost constraints. Furthermore, continuous renal replacement therapy (CRRT) over intermittent dialysis since far more toxins can be removed from the blood using CRRT seven days a week, twenty-four hours a day. Some advantages of CRRT include an expected decrease rate of morbidity and mortality, a decrease in the amount of medications required, a decrease in fluid intake and dietary restrictions, and numerous improvements in the quality of life of the ESRD patients

Existing CRRT machines are large, heavy machines adapted to provide around the clock dialysis, hemofiltration or a combination of both to individual patients. The existing CRRT machines are cumbersome and must be hooked to electrical outlets and several feet of tubing. In addition, these machines require a continuous supply of gallons of fresh water to create dialysate fluid. Further, a patient must remain connected to the existing heavy and cumbersome CRRT machine for many hours, limiting his or her ability to perform normal every day activities.

An additional problem with existing dialysis machines, is that frequent reconnection to the machine requires accessing blood flow by puncturing an arteriovenous shunt. These shunts only last for limited periods of time and are subject to infection, clotting and other complications that result in numerous hospitalizations and repeated surgical interventions.

Patients that use catheters for connection to dialysis devices have an especially high rate of clotting and infection. As a result, these patients also have significantly high rates of hospitalization, morbidity and mortality as compared to those using native fistulas. A wearable CRRT device or wearable artificial kidney (WAK), may provide a solution to raising the quality of life for chronic dialysis patients by increasing the dialysis time from an intermittent 12 hours per week to a continuous, or near continuous, 168 hours of dialysis per week. However, a CRRT device or WAK requires a catheter for blood access and thus may also have an elevated risk of infection and clotting. In order to overcome or decrease the risks of infection, clotting and other related disadvantages, what is needed is a specific detailed protocol on how the CRRT device or WAK should be installed in a patient. Also, there should be a method for monitoring and servicing a CRRT device or WAK (hereinafter CRRT device) in order to prevent or mitigate the risks of infections, such as bacteremia and sepsis, clotting, and maintaining the device in good functioning condition. In addition, there is a need for special care, a method and protocol to assure that the dialysate regenerating mechanism is kept highly functional so that toxins can be continuously removed from a patient's bloodstream and not transferred back into the patient so that a maximum benefit for the patient is preserved.

### SUMMARY OF THE INVENTION

Further applicability of embodiments of the present invention will become apparent from a review of the detailed description and accompanying drawings. It should be understood that the description and examples, while indicating preferred embodiments of the present invention, are not intended to limit the scope of the invention, and various changes and modifications within the spirit and scope of the invention will become apparent to those skilled in the art.

### BRIEF DESCRIPTION OF THE DRAWINGS

The present invention will become more fully understood from the detailed description given below, together with the accompanying drawings, which are given by way of illustration only, and are not to be construed as limiting the scope of the present invention. In the drawings:

FIG. 1 is a perspective view of the wearable CRRT device worn around the waist of a dialysis patient according to the present invention.

FIG. 2 is a front view of the wearable CRRT device of FIG. 1 after being detached from the dialysis patient.

FIG. 3 is a perspective view of the dialyzer section of the wearable CRRT device according to the present invention.

FIG. 4 is a perspective view of the additive pump and dialyzer sections of the wearable CRRT device according to the present invention.

FIG. 5 is a cross-sectional view of a first embodiment of a dialyzer of the wearable CRRT device according to the present invention.

FIG. 6 is a cross-sectional view of a second embodiment of a dialyzer of the wearable CRRT device according to the present invention.

FIG. 7 is a top view of a casing of a dialyzer of the wearable CRRT device according to the present invention.

FIG. 8 is a perspective view of a first embodiment of the sorbent section of the wearable CRRT device according to the present invention.

FIG. 9 is a perspective view of a second embodiment of the sorbent section of the wearable CRRT device according to the present invention.

FIG. 10 is a perspective view of a variation of the second embodiment of the sorbent section of the wearable CRRT device according to the present invention.

FIG. 11 is a top view of a casing of a sorbent device of the wearable CRRT device according to the present invention.

FIG. 12 is a diagram of an exemplary embodiment of the wearable CRRT device.

FIG. 13 is a flow chart depicting the method or protocol for installing and servicing an exemplary CRRT device.

### DETAILED DESCRIPTION OF THE PREFERRED EMBODIMENTS

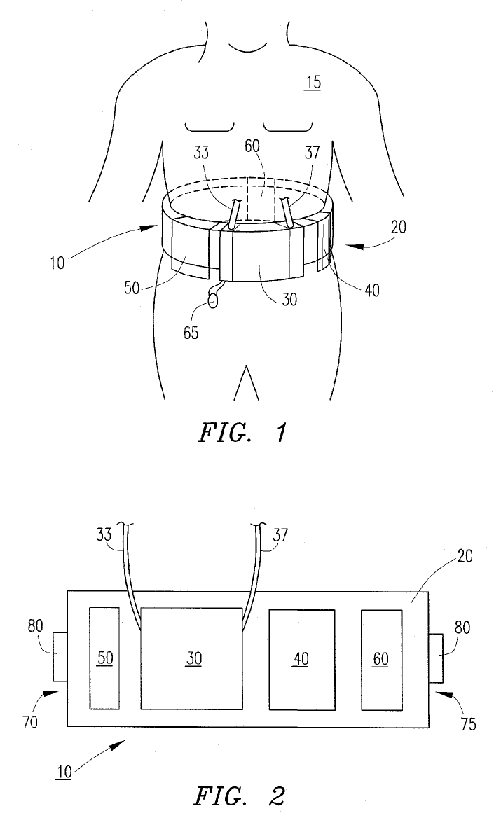

Referring to FIGS. 1 and 2, a continuous renal replacement therapy (CRRT) device 10 is adapted to be worn about a portion of the body of a dialysis patient 15. The CRRT device 10 includes a belt 20 that is divided into a number of sections comprising: a dialyzer section 30 including a blood inlet tube 33 leading from a blood vessel and a blood outlet tube leading to a blood vessel; a sorbent section 40; one or more additive pumps 50; and an electronic control section 60, which includes a microprocessor and batteries to power device 10.

As best seen in FIG. 2, the belt 20 includes a pair of end portions 70, 75, which are secured together by a conventional belt fastener 80 such as a buckle, snaps, buttons or hook and loop fasteners. Although the CRRT device 10 depicted in FIG. 1 is worn about the waist of the patient 15, it should be understood to those of ordinary skill in the art that the device 10 may, alternatively, be worn about other portions of the patient's body, such as over a shoulder of the patient, for example.

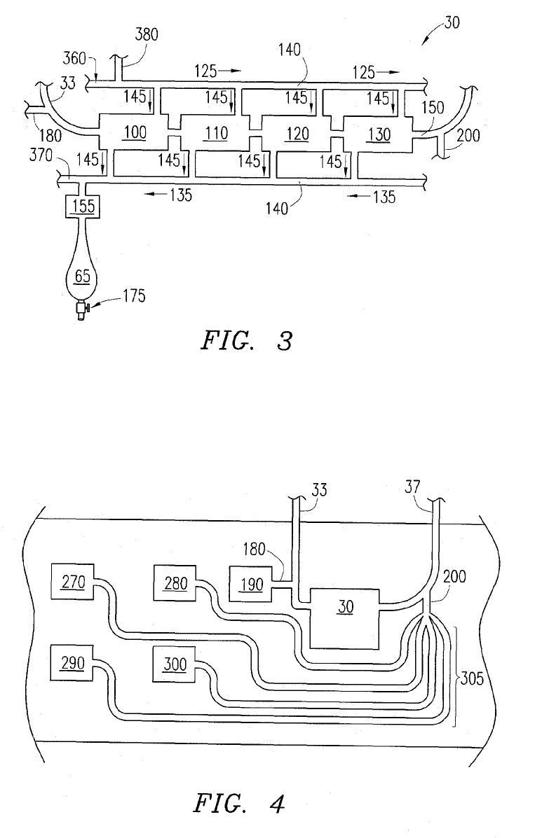

Referring to FIG. 3, the dialyzer section 30 of the belt 20 includes a plurality of miniaturized dialyzers 100, 110, 120, 130 that utilize dialysate fluid 140 to remove impurities from the blood 150 of the patient 15. The number of dialyzers 100, 110, 120, 130 in the plurality of dialyzers 100, 110, 120, 130 may be varied to reflect different dialysis prescriptions. As best seen in FIG. 3, the plurality of dialyzers 100, 110, 120, 130 are connected in series, whereby a conventional pump forces the patient's blood 150 through a blood inlet tube 33, through the dialyzers 100, 110, 120, 130 and into blood outlet tube 37. It should be understood to those of ordinary skill in the art that the dialyzers 100, 110, 120, 130 could also be connected in parallel without departing from the scope of the invention.

During dialysis, the dialysate is pumped in the opposite direction of the blood flow using a conventional pump (not shown) as indicated by arrows 125, 135, 145. Spent dialysate 140 flows toward sorbent section 40 through spent dialysate tube 370. Excess fluid is removed from the spent dialysate 140 through another pump 155 and into a waste receiver 65, which is to be periodically emptied by the patient via tap 175. A microprocessor in the electronic section 60 determines the rate and amount of fluid removal through pump 155.

With further reference to FIG. 3, the blood inlet tube 33 may include a side port 180 through which anticoagulant is pumped into the blood by anticoagulant pump 190. Typical anticoagulants are infused into the blood 150 include, but are not limited to, heparin, prostacyclin, low molecular weight heparin, hirudin and sodium citrate. As best seen in FIG. 4, the blood outlet tube 37 includes a side port 200 for the infusion of additives, which are forced into the blood 150 from a plurality of additive pumps 270, 280, 290, 300. Piston, suction, piezo, micro, or very small roller pumps can be employed for this purpose. Such pumps may all be classified as micropumps. Each additive pump 270, 280, 290, 300 forces a controlled amount of respective additive into the blood 150, wherein the rate of infusion of each additive is controlled electronically by the microprocessor in the electronic control section 60. An additive pump may also force controlled amounts of an additive into the dialysate. In a known manner, a physician can use the electronic control section 60 to set the rate of infusion for each additive to correspond to a predetermined dose for each additive. Since the additives may or may not be mixed together prior to infusion in the blood 150, they have separate circuits 305. Typical additives include, but are not limited to, sodium citrate, calcium, potassium and sodium bicarbonate.

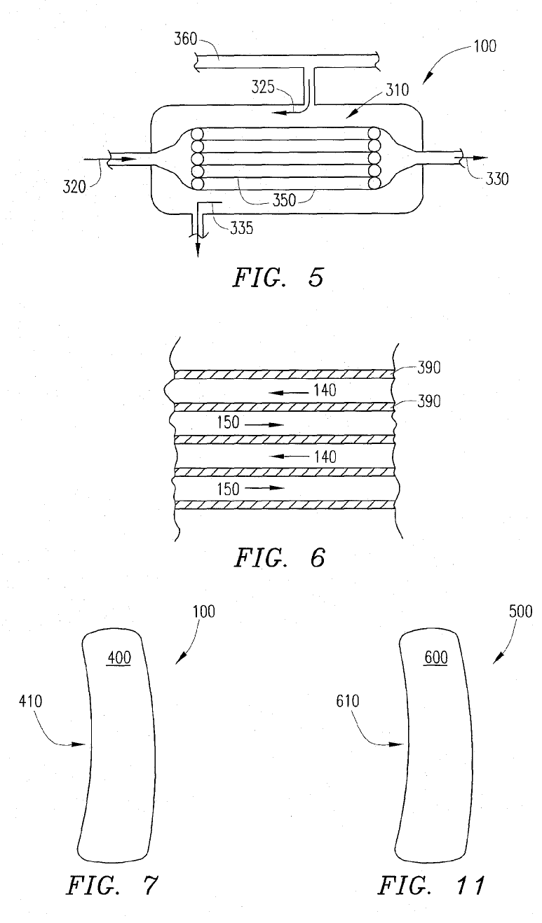

Referring to FIG. 5, in a first dialyzer embodiment, each dialyzer 100, 110, 120, 130 is a conventional dialyzer comprising a plurality of cylindrical hollow fibers 310 through which the blood 150 is circulated. As indicated by arrows 320, 330, the dialysate fluid 140 is circulated around exterior walls 350 of the hollow fibers 310 in a direction across the blood flow inside the hollow fibers 310 as indicated by arrows 325, 335. The exterior walls 350 of the hollow fibers 310 are semiporous so that impurities can be moved from the blood 150 and into the dialysate 140. Fresh dialysate 140 flows from the sorbent section 40 through a dialysate inlet tube 360 and into the series of dialyzers 100, 110, 120, 130. The spent dialysate 140 then flows out of the series of dialyzers 100, 110, 120, and 130, through a spent dialysate outlet tube 370 and into the sorbent section 40. The dialysate inlet tube 360 includes a side port 380 (shown in FIG. 3) for the infusion of additives, which can be forced into the blood 150 via the aforementioned additive pumps 270, 280, 290, 300, whereby the rate of infusion is controlled electronically by the microprocessor in the electronic control section 60. Referring to FIG. 6, in second dialyzer embodiment, each dialyzer 100, 110, 120, 130 comprises a plurality of parallel sheets 390 of semiporous material, wherein the dialysate fluid 140 is circulated on one side of the parallel sheets 390 and the blood 150 circulates in the direction on the other side of the parallel sheets 390.

Referring to FIG. 7, each dialyzer 100, 110, 120, and 130 is a miniature dialyzer having a rigid or flexible casing 400 that may be adapted to conform to the body contour of a patient. In addition, the body-side wall 410 of each casing 400 is concave to further correspond to bodily curves of the user. The casing 400 can, be made of any suitable material having adequate flexibility for conformance to the portion of the body to which it is applied. Suitable materials include, but are not limited to, polyurethane and poly vinyl chloride.

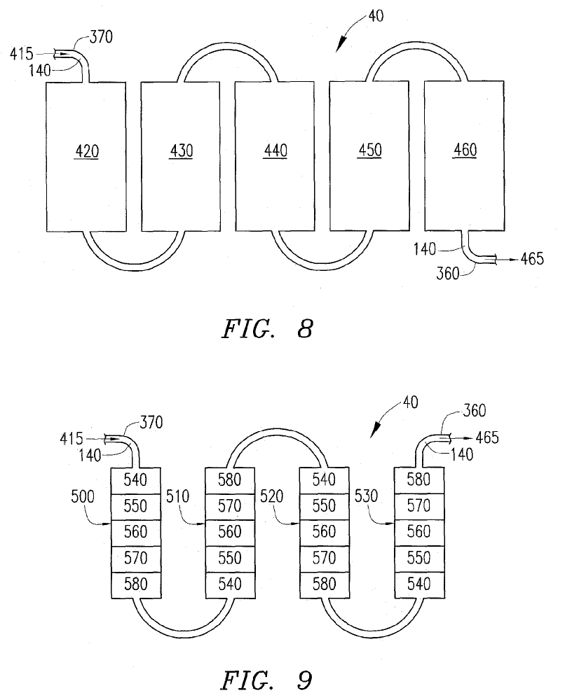

Referring to FIG. 8-10, in the sorbent section 40, as indicated by arrow 415, spent dialysate 140 flows from the dialyzer section 30 through spent dialysate tube 370 and into a plurality of sorbent devices 420, 430, 440, 450, 460. As indicated by arrow 465, the regenerated dialysate 140 then flows through tube 360 and back into the dialyzer section 30. Preferably, the sorbent devices 420, 430, 440, 450, 460 comprise a series of sorbent cartridges 420, 430, 440, 450, 460 for regenerating the spent dialysate 140. By regenerating the dialysate with sorbent cartridges 420, 430, 440, 450, 460, the exemplary CRRT device 10 requires only a small fraction of the amount of dialysate of a single-pass hemodialysis device. Importantly, each sorbent cartridge 420, 430, 440, 450, 460 is a miniaturized sorbent cartridge 420, 430, 440, 450, 460 containing a distinct sorbent.

Referring to FIG. 8, in a first embodiment of the sorbent section 40, there are five sorbent cartridges 420, 430, 440, 450, 460 including an activated charcoal cartridge 420, a urease cartridge 430, a zirconium phosphate cartridge 440, a hydrous zirconium oxide cartridge 450 and an activated carbon cartridge 460. Those of ordinary skill in the art will recognize that these sorbents are similar to the sorbents employed by the commercially available Recirculating Dialysis (REDY) System. However, in the REDY System, the sorbents are layers of a single cartridge. By contrast, the sorbents of the present invention are each part of a distinct sorbent cartridge 420, 430, 440, 450, 460 such that each cartridge 420, 430, 440, 450, 460 may, conveniently, be replaced and disposed of independently of the other cartridges 420, 430, 440, 450, 460 if so desired. As one of ordinary skill in the art would understand, activated charcoal, urease, zirconium phosphate, hydrous zirconium oxide and activated carbon are not the only chemicals that could be used as sorbents in the present CRRT device 10. In fact, any number of additional or alternative sorbents could be employed without departing from the scope of the present invention.

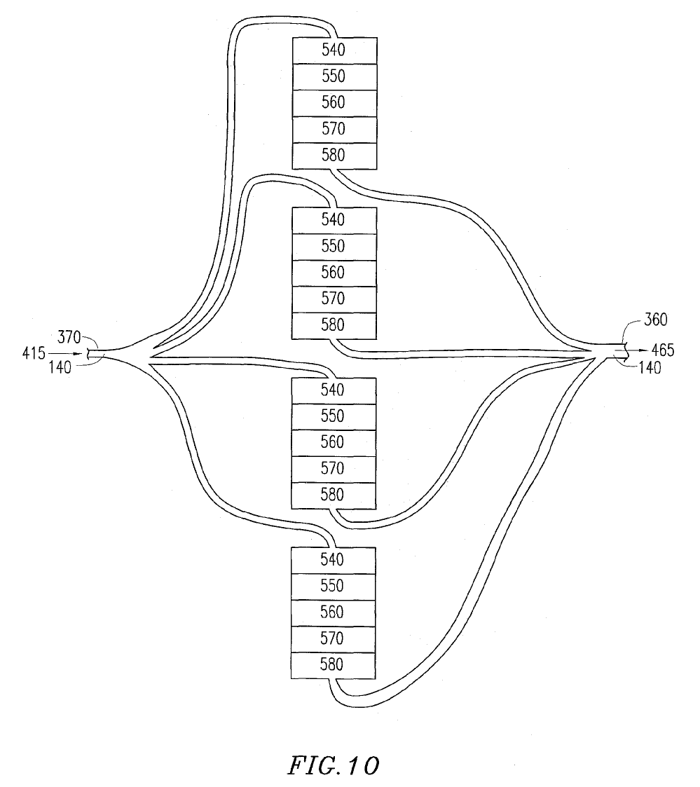

Referring to FIGS. 9 and 10, in a second embodiment of the sorbent section 40, there are a plurality of sorbent cartridges 500, 510, 520, 530, wherein each cartridge 500, 510, 520, 530 includes a plurality of sorbent layers 540, 550, 560, 570, 580: an activated charcoal layer 540, a urease layer 550, a zirconium phosphate layer 560, a hydrous zirconium oxide layer 570 and an activated carbon layer 580. The cartridges 500, 510, 520, 530 may be in series as depicted in FIG. 9 or may be in parallel as depicted in FIG. 10. In this embodiment, the number of sorbent devices may be varied to correspond with different dialysis prescriptions.

Referring to FIG. 11, each cartridge 500, 510, 520, 530 is a miniature cartridge having a flexible or curved casing 600 adapted to conform to the body contour of the patient. In addition, the body-side wall 610 of each casing 600 is concave to further correspond to bodily curves. The casing 600 can be made of any suitable material having adequate flexibility for conformance to the portion of the body to which it is applied. Suitable materials include, but are not limited to, polyurethane and poly vinyl chloride.

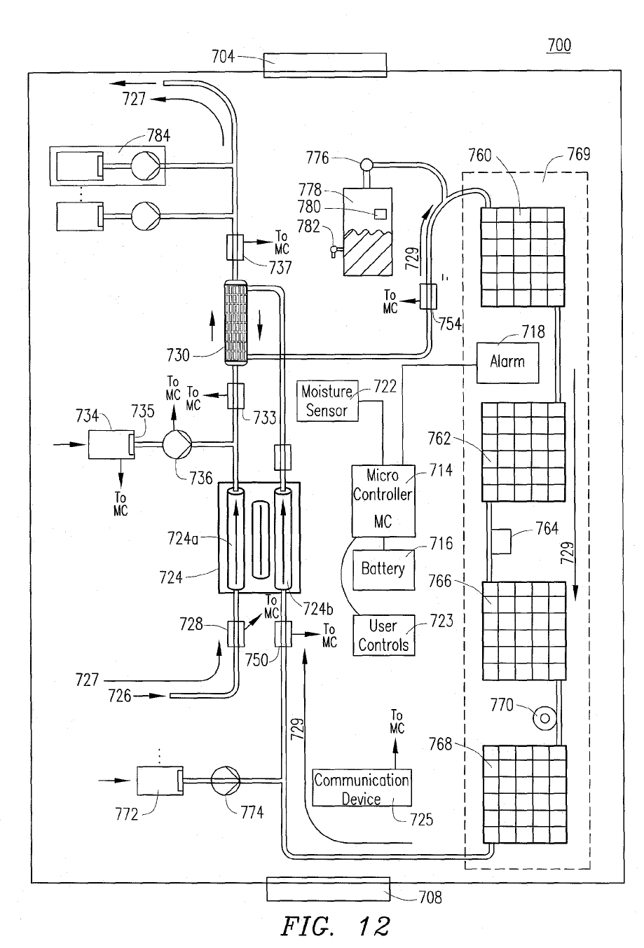

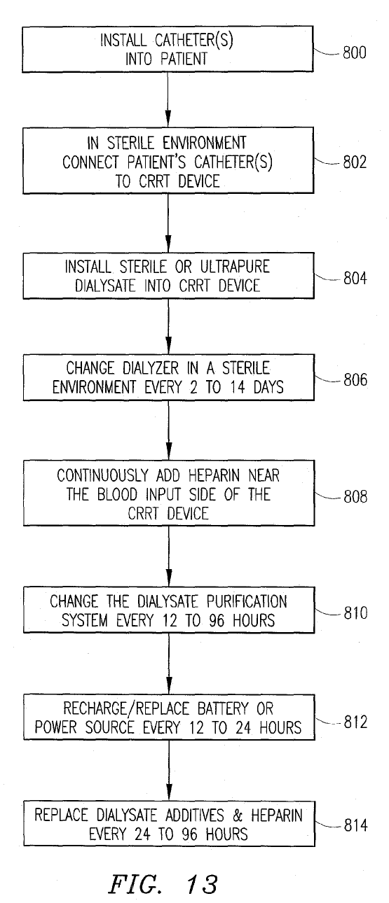

Referring to FIG. 12, another exemplary embodiment of a wearable CRRT device is depicted. The wearable CRRT device 700 is built into, or is part of, a patient wearable strap, belt or other wearable apparatus 702. The belt 702 may include a pair of endportions 704, 708 that are adapted to be secured together by a fastening means (not specifically shown). The endportion/fastening means 704, 708 could be any number of fastening devices suitable to secure the ends of the belt or strap together, but not limited to snaps, button, buckles, clips, laces, hook and loops, zippers, clasps, etc. An embodiment of a CRRT device may be envisioned to be the shape of an ammunition or military style supply belt; it could also be the shape of a waist-pack. An exemplary wearable CRRT device 700 is worn by a patient either over or under other clothing.

A microcontroller 714 is utilized to control and monitor various aspects of the wearable CRRT device 700. The microcontroller 714 is preferably a low or very low power microcontroller, but may be substantially any microcontroller adapted to operate in an exemplary wearable CRRT device 700. One of the many functions of the microcontroller 714 has is to monitor the battery 716. An exemplary CRRT device 700 will operate continuously for at least 5 to 10 hours using less than 10 continuous watts of power. And preferably less than 3 continuous watts of power. Embodiments of the invention weight less than 10 lbs and preferably less than 5 lbs when operating.

The battery or batteries 716 are removably installed in the wearable CRRT device 700. The battery 716 may be rechargeable and may be recharged while remaining in the wearable CRRT device 700 via a charging device (not shown) or when disconnected from the wearable CRRT device 700. Preferably the battery 716 can store enough energy to power a wearable CRRT device 700 for at least five (5) or more hours of continuous uninterrupted device operation. The microcontroller, by itself, or via additional circuitry, monitors the charge status of the battery 716. If the microcontroller 714 determines that the battery 716 is low on charge or has less than an estimated predetermined amount of operating time left (e.g., one hour left), the microcontroller 714 may trigger an alarm condition via alarm circuit 718. Alarm circuit 718 may provide any combination of an audio, visual, or physical alarm. The physical alarm signal may include vibrations or small tingle-style shocks to the patient. An alarm condition or warning may be displayed on the display 720 using liquid crystal, light emitting diode or other low power display technology. An alarm condition may also shut down all or predetermined parts of an exemplary wearable CRRT device 700.

A moisture sensor 722 may be installed and be in electrical communication with the microcontroller 714. The moisture sensor 722 is used to detect high humidity, condensation, or liquid present inside the packaging or covering over (not specifically shown) the wearable CRRT device 700. The packaging or covering over an exemplary CRRT device 700 may be a plastic, cloth, rubberized, poly-product, or other suitable material. The covering may cover a portion of the wearable CRRT device 700 and allow access to various parts of the device such as the display 720 and user/doctor controls 723.

High humidity, condensation or the presence of liquid inside or around a wearable CRRT device 700 may be indicative of patient blood leakage, dialysate leakage or other fluid leakage. Upon sensing moisture, the moisture sensor 722 provides a signal to the microcontroller 714 and an alarm is triggered via the alarm circuit 718. Furthermore, the pump 724 may be turned off by the microcontroller 714 to help minimize further blood, dialysate or other fluid loss. The microcontroller may shut down the micropumps (to be discussed later) also. The microcontroller 714 may also prompt an onboard communication device 725 to contact medical help or another entity for medical assistance. The communication device may comprise a paging wireless phone or other mobile communication circuitry. The communication device 725 may also be able to provide the geographic location of the exemplary wearable CRRT device 700.

The pump 724 is an electric pump. The pump 724 may be two pumps 724a and 724b. The two pumps 724a and 724b may each operate off the same or separate electric motors. The pumps 724a and b are powered by the regular or rechargeable battery 716. Furthermore, the microcontroller 714 can be used to adjust various pumping variables. Potential adjustable pumping variables include, but are not limited to, adjusting the pump stroke, volume-per-stroke, speed, torque, pumping rate (i.e., number of pump cycles per minute), pump pressure, pump pressure differential between the input and output of the pump, and pump pause and cycle times.

An exemplary wearable CRRT device 700 has two fluid circuits: a blood circuit 727 and a dialysate circuit 729. A dual channel pulsatile pump 724 may be used in an exemplary embodiment. A pulsatile pump, in general, has a rubberized cartridge for each channel. A cartridge has an input valve at an input side of the cartridge and an output valve at an output end of the cartridge. FIG. 12 depicts a single direction, dual pulsatile pump 724. A dual direction, dual pulsatile pump may also be utilized. A dual direction channel pump is preferred in order to decrease bending of the tubing used in the fluid circuits.

The motor and transmission within the pulsatile pump presses the rubberized, tubular portion of the cartridge. The pressing of the cartridge squeezes and evacuates the contents of the cartridge out of the output valve. As the pump motor spins and causes the mechanics of the pump to release pressure from the rubberized portion of the cartridge, the output valve closes and the input valve opens to allow fluid (blood or dialysate) to enter the cartridge so that the fluid can be squeezed out the output valve in the next pump cycle. The input and output valves are one-way valves allowing fluid flow in a single direction through the cartridge. Other configurations of a pulsatile pump are also available. An exemplary pump 724a, 724b provides a blood flow rate of between about 15 to 100 ml/min (pulsatile). The approximate dimensions of an exemplary dual-pulsatile pump 724 are 9.7×7.1×4.6 cm with a weight of less than 400 grams. An exemplary pulsatile pump uses less than 10 watts of energy and may provide a low battery power and a pump occlusion alarm signal to the microcontroller 714. A lower power pulsatile pump using 5 or less watts may also be used.

The pulsatile pump can be tuned such that the pulses, or cycles, of the two pulse chambers are in phase, 180° out of phase or any predetermined number of degrees out of phase in order to utilize the pulses of the pump to aid in maximizing the dialysis process occurring in the dialyzer 730. The opposite directional flows of blood and dialysate through the dialysate may become more efficient at different phase settings of the pumps 724a and b.

Other types of pumps 724 can be successfully used or incorporated into embodiments of the wearable ultrafiltration device. Two separate pumps may also be used. Such other types of pumps include, but are not limited to, a shuttle pump, a piston pump, a roller pump, a centrifuge pump, a piezo electric pump, or other conventional pumps. Whatever pump is utilized, the pump(s) 724 should have a manually or electrically adjustable flow rate ranging somewhere between 20 ml/min and 120 ml/min.

Usage of an out of phase pump cycle between the two pumps is significant. For example, having the pulsations of the blood and dialysate pump circuits being a half cycle out of phase are significant because the pump phase difference helps to maximize the transmembrane gradient across the dialysis membrane and invert the direction of the gradient with each alternating pulsation thereby creating a “push-pull mechanism” and a major convective mass transfer from the blood to the dialysate and vice versa. The result being an efficient clearance of solutes from the blood. The phase difference may vary and may also include a beat frequency between the pulsations of the pumps. In other words, the pumps may operate at the same pumping frequency, but out of phase or may operate at different or differing pumping frequencies.

The microcontroller 714 may display pump status or other pump related information on the display 720. User controls 723, being buttons, switches, slide controls, knobs, connectors, or infrared receiver (not specifically shown) may be used to enable a patient, physical, nurse, technician or computer based device to adjust various settings and controls on an exemplary ultrafiltration device 700. Furthermore, the communication device 725 may be utilized to receive control settings and send information via paging or other telecom communication channels. For example, the adjustments to the pump 724 pump rate, torque, valve opening size, output pressure, flow rate, rpm, and on/off may all be monitored or controlled via the user interface 723 or the communication device 725.

Discussing the exemplary blood circuit 727 first, blood from the patient enters the blood circuit 727 via the blood inlet tube 726. An input blood pressure transducer 728 measures the input blood pressure and provides an input blood pressure signal to the microcontroller 714 (connection to microcontroller not specifically shown). The input blood pressure may be an average pressure of the blood prior to entering the pump 724a. The blood is then pumped through the pump 724a.

After the blood passes through the main pump 724a, it continues in the blood circuit 727 via the blood inlet tube 726. An input blood pressure transducer 728 measures the input blood pressure and provides an input blood pressure signal to the microcontroller 714 (connection to microcontroller not specifically shown). The input blood pressure may be an average pressure of the blood prior to entering the pump 724a. The blood is then pumped through the pump 724a.

After the blood passes through the main pump 724a, it continues in the blood circuit 727. A reservoir 734 containing a blood thinner or anticoagulant such as heparin or another acceptable anticoagulant additive is connected to the blood circuit via a micropump 736. The micropump 736 provides the fluid contents of the reservoir 734, in a measured continuous or non-continuous manner, to the blood circuit 727 prior to the dialyzer 730. (It is possible to connect the reservoir 734/pump 736 combination to the blood circuit before the pump 724a.) The micropump 736 is a type of pump that can pump microscopic or miniscule amounts of fluid each minute. A micropump, in general, may pump fluid at a rate ranging from 0.1 to 400 ml/hr (milliliters per hour). A micropump requires from about 1 to 500 milliwatts to operate. There are, at present, various pumps that can be considered micropumps including, but not limited to, a piezoelectric pump, a solenoid pump, a micro-piston pump, a peristaltic pump, a nanotechnology related pump, microtechnology/micromachined pump, syringe style pump, roller pump, centrifuge style pump, or diaphragm style pump.

The blood thinner and/or anticoagulant may be mixed or combined with the blood in the blood circuit at any point between the inlet of blood inlet tube 726 and the blood input side of the dialyzer 730. In some embodiments of the invention, the anticoagulant infusion may be avoided by the administration of other anticoagulants to the patient. Such other anticoagulants include, but are not limited to, Warfarin, low molecular weight heparin, hirudin, or argatroban.

The reservoir 734 may have a fluid level sensor 735 or other type of sensor to sense the amount of fluid available in the reservoir 734. The sensor 735 provides a signal to the microcontroller 714 indicating an amount of fluid in the reservoir 734. The microcontroller 714 sends an alarm signal to the alarm circuit 718 if the fluid level or fluid amount in the reservoir 734 is below a first predetermined amount or volume. The microcontroller 714 may also turn the ultrafiltration device 700 off if the fluid level in reservoir 734 is at the first predetermined level or below the first predetermined level and at a second predetermined level.

The combination of the reservoir 734 and the micropump 736 infuse the blood thinner or anticoagulant into the blood flowing in the blood circuit 727. Again, the thinner or anticoagulant is infused into the blood prior to the dialyzer (or blood filter) 730 (in some embodiments prior to the blood pump 724a) in order to help minimize the potential of blood clots in the blood filter 730 and perhaps in the blood pump 724a.

A second pressure transducer 733 senses the pressure in the blood circuit after the blood pump 724a, but before the dialyzer 730. The pressure reading is supplied to the microcontroller (MC) 714 which monitors such readings. In alternate embodiments of the invention these transducers may be unnecessary.

A dialyzer 730, shown as a single dialyzer, can be a single or multiple dialyzers as discussed earlier. The dialyzer(s) may take the form of a cartridge that can be “clicked” or inserted into and out of the blood/dialysate circuits by a doctor, nurse or technician. The dialyzer may comprise from 0.2 to 1 sq. meters of dialyzing surface area. During dialysis the blood circuit 727 flows in the opposite direction as the dialysate circuit 729 in order to help maximize the dialysis process. Furthermore, the pulsing of the pumps 724a and b may, either in phase or out of phase, also aid in maximizing the dialysis processes.

The blood, after being dialyzed in the dialyzer 730, exits the dialyzer 730 and flows through a third pressure transducer 737. The third pressure transducer 737 provides a pressure signal to the microcontroller. The combination of the first, second and third transducers provide differential pressure measurements that are analyzed by the microcontroller 714. For example, if the pressure differential across the dialyzer 730 is too high it may mean, among other things, that the dialyzer 730 has a clot in it or is being operated at too high a blood flow. As a result, an alarm situation can be initiated or the blood pump 724a pumping rate or torque can be adjusted via microprocessor control. If the pressure at a transducer drops below a predetermined pressure it may be an indication of a fluid leak or that air is in the blood circuit 727. The microcontroller 714 may shut down all or predetermine parts of the wearable CRRT device 700 in response to pressure measured below a predetermined level.

The blood returns to the patient via the blood outlet tube 740. As shown in FIG. 4, a sideport 200 can be incorporated so that additional electrolytes, drugs, blood additives, vitamins or other fluids can be added to the blood in the blood circuit 727 via a reservoir/micropump combination prior to the blood being returned to the patient via the blood outlet tube 740.

Referring still to FIG. 12, the exemplary dialysate circuit will now be discussed. A fourth pressure transducer 750 measures the dialysate pressure at the input side of the dialysate pump 724b and provides the pressure reading to the microcontroller 714. The dialysate pump 724b, like the blood pump 724a is preferably part of a dual pump device 724 described above, but may be a separate pump device.

Cleaned, fresh dialysate from the sorbent filters 769 flows in the dialysate circuit 729 through the dialysate pump 724b. The dialysate pump 724b can pump dialysate at a flow rate ranging from near zero to 150 ml/min. The exemplary normal operating flow rate of the dialysate pump is between 40 and 100 ml/min.

Embodiments of the wearable CRRT device 700 are designed to operate using less than one liter of dialysate. Embodiments preferably only require 300 ml to 400 ml in the closed dialysate fluid circuit 729 to operate. An embodiment designed for a young adult or child may operate with about 100 to about 300 ml of dialysate. The combination of dialysate and filters 769 allow an embodiment to circulate dialysate for at least 24 hours before a filter requires replacement. Furthermore, because less than a liter of dialysate is all that is needed in the closed dialysate circuit 729, sterile or ultra-pure dialysate can be economically used in exemplary embodiments of the wearable CRRT device 700.

In normal or large dialysis machines it is common to use about 90 liters of dialysate per patient per run. Generally, due to of the amount of water required to create the dialysate, filtered water, rather than ultra-pure water, is used. Filtered water is much less expensive than ultra-pure or sterile water. Filtered water that is used in dialysis machines is allowed to have some bacteria in it. The bacteria are larger than the size of the pores in the membranes used in the dialyzer 730. Since the bacteria is larger than the pore size, the bacteria cannot cross the membrane and get into the blood.

Conversely, medical research has provided some results that are uncomfortable with the use of non-sterile dialysate (dialysate containing filtered water, bacteria, toxins, or micro organisms). The micro organisms and bacteria create waste products, toxins or poisons in the dialysate. The waste products from the bacteria can cross the dialyzer pores and get into the patient's blood while the actual bacteria cannot. Such toxins are referred to, in some cases, as endotoxins. The endotoxins that pass from the dialysate to the blood can have a negative effect on the patient's health. The endotoxins can make the patient sick.

Since exemplary embodiments of the wearable CRRT device 700 require less than one liter of dialysate it is economically feasible to use ultra-pure, toxin free, or sterile water when making the dialysate. The use of ultra-pure, toxin free, or sterile water with completely wearable renal replacement devices is a new concept.

The dialysate exits the dialysate pump 724b, passes by another pressure transducer 752, which measures the dialysate pressure on the input side of dialyzer 730. The dialysate circuit 729 moves the dialysate into the dialyzer 730 such that the dialysate preferably moves in a direction opposite to the flow of blood through the dialyzer. While the dialysate is in the dialyzer to the dialysate 730, waste products and toxins in the blood pass through the membranes of the dialyzer to the dialysate thereby cleaning the patient's blood.

The dialysate exits the dialyzer 730 and flows through another pressure transducer 754. The pressure transducer 754 on the output side of the dialyzer 730 sends a signal to the microcontroller 714 indicating the pressure of the dialysate. The pressure may help indicate a clogged dialyzer, a leak or other emergency condition.

The dialysate circuit 729 takes the used, toxin or contaminant containing, dialysate to the first of a series of dialysate filters 769. The filters may filter or react with predetermined substances in the dialysate in order to recycle the dialysate for continued use in the dialysate circuit.

In an exemplary embodiment, the first filter 760 contains urease. The urease filters the used dialysate and further functions to break down urea that was removed from the blood in the dialyzer 730. When urease breaks down urea at least two unwanted bi-products are created. Generally, the two bi-products are ammonium (ammonia) and carbon dioxide.

The dialysate with the ammonia and carbon dioxide exit the first filter 760. The urea is substantially removed from the dialysate, but the ammonia and carbon dioxide need to be removed from the dialysate also. The dialysate, ammonia, and carbon dioxide enter the second filter 762. The second filter 762 contains a compound containing zirconium or zirconium phosphate (i.e., ZrPx). The zirconium in the second filter 762 captures the ammonia. It is understood by those having ordinary skill in the art of dialysis chemistry that various chemicals and derivations thereof can be utilized to achieve the same or similar results.

The zirconium filter, the second filter 762, will eventually become saturated with ammonia. The zirconium filter, when becoming saturated with ammonia, will become less efficient at removing ammonia from the dialysate. It is not advantageous to allow ammonia or ammonium to circulate through the dialysate circuit 729. Thus, in an exemplary wearable CRRT 700, a sensor 764 is placed in the dialysate circuit 729 to sense a presence of ammonia in the dialysate. The sensor 764 may be a pH sensor, an ammonia specific sensor, or a conductivity sensor. If an ammonia sensor is used it will sense whether a predetermined amount of ammonia is present in the dialysate. If a pH sensor is used, it would sense whether the ph of the dialysate has become a predetermined amount more alkaline than normal. When ammonia is present, the dialysate becomes more alkaline. It is noted that depending on the actual chemicals and absorbents used in the filters, the dialysate may become more acidic and as such a sensor would be used to sense the same. If a conductivity sensor is used, it will sense the conductivity changes of the dialysate.

The sensor 764 is in electrical communication with the microcontroller 714. If the signal read by or provided to the microcontroller 714 from the sensor 764 indicates that the second filter 762, the zirconium filter, is not adsorbing a majority or a predetermined amount of the ammonia in the dialysate, then an alarm condition is triggered by the microcontroller 714. The alarm condition would instruct the user that one or more filters (cartridges) need to be replaced. The alarm condition may also shut down predetermined functions of the wearable CRRT device 700. For example, one or more pumps 724 may be shut down or the pump rate of one or more pumps and micro pumps may be slowed. Slowing the pump rate may increase the amount of ammonia adsorbed by the zirconium based filters in the sorbent filter section 769.

The sensor 764 that is used to sense the presence of ammonia in the dialysate is placed after the second filter 762 containing the zirconium phosphate. The sensor 764 may be placed after the third filter 766, that contains hydrous zirconium oxide or the fourth filter 768 which is a carbon filter. One or more sensors in the dialysate circuit will sense pressure, pH, ammonia, flow rate, temperature or other physical attributes. A sensor will provide a signal to the microcontroller indicating that the dialysate circuit needs maintenance.

The third exemplary filter 766 is a hydrous zirconium oxide (ZrOx) filter which may further remove contaminants and ammonia from the dialysate. A bubbler, degasser, or valve device 770 may be part of a filter (i.e., 762, 766 or 768) or be a separate element, as shown, removes air, carbon dioxide and other gas bubbles from the dialysate. It is important that a limited amount of gas bubbles go through the dialyzer 730. As such a bubbler 770 (one or more) should be positioned prior to the pump 724b, but after the filter or filters that may cause gas bubbles to form in the dialysate.

The fourth exemplary filter 768 contains carbon and is used to further clean the dialysate of impurities via adsorption. The filters, as discussed previously, are preferably designed as filter cartridges. Each cartridge can be inserted and removed from the wearable CRRT device 700 by the patient, doctor, technician or nurse. Each filter cartridge 760, 762, 766, 768 may contain layers or combinations of chemicals or adsorbents. In fact, an exemplary embodiment may have a single cartridge filter containing layers of required substances to clean and refresh the dialysate after passing through the dialyzer 730. The filter cartridge(s) may each incorporate a bubbler device or the bubbler 770 may be a separate element in the dialysate circuit 729.

In an exemplary wearable CRRT device 700 the cartridge(s) may be replaced daily or every other day by the patient. Each filter cartridge should weigh less than half a pound dry. The combination of all the cartridges, dry, should weigh less than two pounds total. Each filter cartridge may have inner dimensions of about 4 cm×10 cm×10 cm or provide a volume of about 400 cm³±100 cm³ for each sorbent material. The total volume of all sorbent materials using in whatever quantity, combined, may be between about 400 cm³±2,000 cm³. In an exemplary embodiment a filter cartridge can be changed one a day or less often.

An additive reservoir 772 and micropump 774 may be connected to the dialysate circuit 729 after the filter cartridge(s) 769, but before the pump 724b. Although not specifically shown in FIG. 12 multiple reservoirs 772 and micropumps 774 can be connected to the dialysate circuit 729. The micropump(s) 774 may be any of the micropumps discussed above with respect to micropump 736. Here the micropump(s) 774 and reservoirs 772 may add chemicals and additives into the dialysate in order to prolong the dialysate's ability to act as a dialysate. An exemplary wearable CRRT device 700 may have as little as 300 ml to about one liter of dialysate in the dialysate circuit 729. It is important for the sorbent section 769 to be able to clean and to freshen the dialysate continuously as the dialysate circulates about the dialysate circuit 729.

An exemplary wearable CRRT device 700 may also remove ultrafiltrate or fluids from the patient's blood. The patient's kidneys may not be functioning properly. After the dialysate leaves the dialyzer 730, and preferably before the dialysate enters the filter cartridge(s) 769 (dialysate purification system), ultrafiltrate/dialysate, along with other contaminants and fluids obtained via the dialyzer 730, can be removed from the dialysate circuit 729 via a valve 776 and deposited in a fluid bladder 778. The fluid bladder 778 may hang below the wearable CRRT device 700 (not specifically shown) and be able to store from about 0.1 to 2 liters of fluid. A fullness sensor associated with the fluid bladder 778 is in electrical communication with the microcontroller 714 to enable an alarm condition when the fluid bladder 778 at a predetermined fullness.

The fluid bladder 778 may also be incorporated into the wearable CRRT device 700 as an empty cartridge that is filled via a micropump and valve combination 776. A fullness sensor 780 can aid the microcontroller to determine the fullness of the cartridge bladder 776 will turn off the ultrafiltrate supplying micropump 776 and provide a signal to the user that the cartridge needs emptying. The fluid bladder or cartridge 778 may contain an absorbent material (not specifically shown) for absorbing fluid presented to the bladder 778. The absorbent material may be a cotton, polymer, sponge, compressed material, powder, jell or other material that absorbs fluid and/or limits sloshing in the bladder or cartridge. The bladder may be designed to expand as it fills. The bladder may press against a microswitch 780 (not specifically shown) when it is full thereby providing a signal to the microprocessor 714.

The fluid bladder or cartridge 778 may have a means for emptying the fluid bladder 782 thereon in the form of a cap, stopper, valve, removable inner bladder or otherwise.

Referring back to the blood circuit in FIG. 12, reservoir/micropump combinations 784 (piezo pumps, solenoid pumps, syringe pumps, or other types of very low power pumps.) can be connected to the output side of the blood circuit dialyzer 730, 727. One or more micropumps and fluid reservoirs 784 can be connected. Additional heparin, electrolytes, blood additives, drugs, vitamins or hormones can be added to the dialyzed blood returning to the patient's body. The reservoir/micropump combinations are monitored and controlled by the microcontroller and can be adjusted via the user controls 723, or instructions received via the communication device 725.

Exemplary embodiments of the wearable CRT device can provide therapy from a basic dialysis function to a more complex medical dialysis, ultrafiltration, and medicinal therapy to a patient.

As discussed, there continues to be a growing body of literature indicating that increasing dialysis time, being longer or more frequent dialysis treatments, may be associated with improved outcomes in the treatment of End Stage Renal Disease (ESRD) patients, both in terms of life quality as well as expected morbidity and mortality.

However, the implementation of such modalities of treatment is complicated because of the lack of readily available economic resources to pay for the increased time or more frequent dialysis treatments. Furthermore, even if the money to pay for more dialysis time or treatments was available, there is currently limited additional nursing or technician manpower to deliver much more additional care. In addition, construction of additional facilities would be necessary to accommodate all these additional needs. Given the budgetary constrains of health care budgets in most countries, the chances of any or all of these things occurring is slim. Furthermore, very few dialysis patients are suitable for home self-treatment on non-wearable dialysis devices.

Embodiments of the wearable CRRT device are generally worn on a belt or strap by the patient and can be used for continuous renal replacement therapy twenty-four hours a day, seven days a week. Such embodiments can deliver significantly higher doses of dialysis than the intermittent dosing commonly administered by dialysis facilities today, while at the same time achieving significant reductions in manpower utilization and other medical related costs.

Recently an embodiment of the invention was tested to assess the efficiency and viability of the inventions in a uremic pig model. The efficiency of the exemplary wearable CRRT device was evaluated by achieving the removal of urea, creatinine, potassium, phosphorus and ultrafiltrate in amounts that would normalize the volume status as well as the above chemistries in uremic humans if the device would be worn continuously. Furthermore, the efficiency of the device was tested by achieving dialysis doses that would be equal to or higher than those afforded by intermittent daily dialysis, as measured by creatinine clearance, urea clearance and weekly urea Kt/V.

The exemplary embodiment of the wearable CRRT device used in the test comprised a blood circuit and a dialysate circuit. The blood circuit and dialysate circuit flowed through a small dialyzer that utilized polysulfone hollow fibers. The dialyzing surface area of the dialyzer was about 0.2 meters. The blood circuit had a port for the continuous administration of heparin into the circuit prior to the dialyzer. Both the blood and dialysate were propelled through their requisite circuits via a double channel pulsatile pump powered by replaceable batteries. The dirty or spent dialysate that exited the dialyzer was circulated through a series of filter cartridges containing urease and sorbents similar to those described by Marantz and coworker and widely used in the well known REDY system. Ultrafiltrate was removed by the dialysate circuit via a valving structure. The removed ultrafiltrate was directed to and stored in a plastic bag that was periodically emptied after volume measurement. Sensors connected to a micro pressure sensor monitored various aspects of the exemplary device.

Six farm raised pigs each weighing approximately 150 lbs. were anesthetized and made uremic by surgical ligation of both ureters. Twenty-four to forty-eight hours later the animals were again anesthetized and a double lumen Mahurkar catheter was inserted in a jugular vein. The catheter was connected to the exemplary CRRT device and each animal was dialyzed for eight hours. At the end of the eight hours the animals were euphemized.

Blood samples were drawn from an arterial line inserted in the carotid artery and CBC, urea, creatinine, sodium, potassium, chloride, CO₂, phosphorus, calcium and magnesium were measured. The same chemistries were measured in the dialysate circuit at the input side of the dialyzer and at the exit side of each filter cartridge.

The results of the test experiment were as follows. There were no adverse events observed in the animals during the test experiments. The average blood flow rate in the blood circuit was 44 ml/min and the average dialysate flow rate was 73 ml/min. The relatively low flow rates of the blood circuit and dialysate circuit help mitigate various complications found in some dialysis systems. Modifications can be made to the experimental exemplary CRRT device to allow an increase in the blood flow to range from about 50 to 120 ml/min. The modifications include at least one of increasing the size of the dialyzer, increasing the flow of the dual pump, and adjusting the transmission, gearing and valving of the pump.

The capacity of an exemplary wearable CRRT device to remove fluid steadily from the vascular space in amounts similar to the volume of fluids removed physiologically by normal kidney gives a treating physician the ability to keep a patient euvolemic, regardless of the amount of fluid the patient ingests. Further, the elimination of excess fluid may also result in better control of a patient's hypertension. The sodium concentration in the extracted ultrafiltrate is roughly equal to the sodium concentration of the patient's plasma. Thus, removal of about 0.5 to 3 liters of ultrafiltrate, via an exemplary CRRT device, a day will result in removal of about 10 to 20 grams of salt per day. Removal of sodium from a patient via an embodiment of the invention may contribute to better control of a patient's hypertension, and also result in liberalizing salt intake for ESRD patients. Thereby, perhaps improving a patient's quality of life by increasing the variety of foods a patient can eat. Furthermore, eating a variety of foods may result in improved nutrition for the patient.

Also, the amounts of potassium and phosphorus removed from a patient's blood by an exemplary wearable CRRT device further helps eliminate restrictions on oral intake of both the elements, and the elimination of a need for oral phosphate binders.

The experimental results indicated that the amount of creatinine and urea removed, as well as the high dialysis dose, expressed in both clearances and weekly urea Kt/V would make it feasible to achieve all the benefits of presently provided intermittent daily dialysis doses. The experiment, at the same time, proved a potential for decreasing the use of medical manpower and other costs associated with chronic dialysis.

According to current practices in the dialysis industry, the establishment, or the manipulation of the large scale dialysis units and their blood circuit that is connected to a patient, is done under non-sterile conditions. To date, changes of blood tubing and dialyzers in large scale dialysis machines, as well as small scale machines, are performed in environments that are not sterile. In fact, in environments where existing large scale dialysis units exist, non-scrubbed and non-gowned patients and personnel are allowed in and out of their doors. Dialyzers and blood tubing are connected and disconnected from patient and machine with very little regard for sterility. The exception, of course, is an avoidance of contacting a sterile surface with a non-sterile surface.

It is the norm that personnel in charge of connecting patients to existing dialysis machines may wash their hands before performing their duties, but normally use non-sterile gloves, are not scrubbed, and do not use sterile gowns, caps or masks. Dialysis patients are not required to be masked or gowned, nor do they have to cover their hair. All of these factors make the penetration of bacteria into the blood circuit of present day dialysis devices a very likely event. Such an event may result in catastrophic septic episodes for the patient. To date there are no formal methods or protocols designed and directed to help prevent or mitigate the risk of bacterial penetration of the blood circuit in a dialysis system.

As such, it is important since an exemplary CRRT device (WAK) is worn on a substantially continuous basis by a patient, that all manipulation, connection or disconnection of the blood circuit or any of its components (blood tubing, dialyzer, blood pump, etc.) must be performed in a sterile environment observing all the standard rules of sterile technique. As such, manipulation, connection, or disconnection of the blood circuit of an exemplary CRRT device would be performed by a nurse or a properly trained technician, who has carefully scrubbed their hands, wears a sterile gown, a head cap, a mask, and sterile gloves. In addition, the patients will wear a mask and hair cap, as well as a sterile gown. Any procedure with an exemplary CRRT device that involves the blood circuit, will take place in a sterile environment room, similar in sterility to an operating room. No person would be allowed in such a room unless they comply with the same sterility requirements as those required in an operating room. Since a CRRT device, once operational, is not expected to require any change or manipulation of the blood circuit for several days (2-14 days), the implementation of this sterile environment protocol for the blood circuit would be practical and economically feasible. Furthermore, it is expected that there would be a reduction of hospitalizations of dialysis patients because of a decreased incidence of infections and associated morbidity resulting in further economic advantageous of using an exemplary CRRT device.

In addition, the currently used dialysis regimes that utilize catheters in a patient, allow for a formation of a stagnant column of fluid in the patient's catheter lumen. Blood does not flow or circulate inside the lumen until the catheter is connected to a dialysis machine again. It is understood in the current regime of dialysis, patients are generally connected to a dialysis machine for a few hours and then disconnected from the machine for 48-72 hours. As such, during the 48-72 hours of blood not flowing through the stagnant column of fluid in the patient's catheter lumen, any bacteria that gets into the blood circuit and remains in the catheter lumen is not reached by leukocytes or antibodies from the patient's body because the blood in the catheter is not circulated through the lumen. Therefore, any such bacterium that is in the lumen of the catheter while the patient is not connected to a dialysis device has ample time and opportunity to colonize and multiply with impunity until the catheter is used again.

Conversely, in embodiments of the present invention and protocol or method of installing and servicing a completely wearable CRRT device, the CRRT device provides continuous blood circulation that does not allow any stagnant column of blood or fluid to form in a lumen of the blood circuit catheter. Therefore, in the unlikely event that bacteria penetrated the blood circuit catheter, such bacteria would be immediately washed out by the continued blood circulation and therefore expose both to antibodies and white cells of the patient. Such bacterium would have no opportunity or time to colonize or multiple significantly. It is expected that this continuous flow in the blood circuit of an exemplary completely wearable CRRT device would mitigate, largely, the risk of bacteremia and sepsis of patients in this particularly high infection risk population.

In existing dialysis systems, clotting in the dialysis catheter lumen presents a major source of blood access failure in the dialysis patient population. At times, clotting of the dialysis catheter lumen requires the physical removal of the clot or its dissolution by the installation of dissolving enzymes, such as, urokynase or Tpa. As discussed above, intermittent use of the dialysis catheter by the dialysis patient, allows for a stagnant fluid column to be present in the catheters lumen. The catheter, which is installed into the dialysis patient, remains without blood circulation or flow for 48-72 hours. If fibrin or platelets are present in the fluid column, then a clot will probably form as platelets aggregate and the fibrin precipitates. Although it is a common practice to flush these catheters with heparin, a non-anticoagulant, at the end of a dialysis session, heparin loses its effectiveness after only a few hours. To date, there is no dialysis method being used that provides for a substantially continuous, 24 hour-a-day, 7 days-a-week flow of heparinized (or other anticoagulant for that matter) blood through a dialysis blood circuit catheter. An exemplary CRRT device provides a continuous flow of heparin into its blood circuit as provided by a micro pump. The flow of heparin is provided near where the CRRT device is connected to the patient's internal catheter wherein the blood enters into the CRRT device from the patient. The continuous flow of heparin into the blood circuit prevents clotting. It is noted that any number of other anticoagulants may be successfully used besides heparin, hirudin or argatroban. Furthermore, the continuous flow of blood through the lumen in the catheter and blood circuit prevents the stagnation of fluid in the lumen that would incite the precipitation of fibrin or the aggregation of platelets that would result in clot formation.

As discussed previously, in existing dialysis machines and using existing dialysis techniques, fresh dialysate is continuously pumped into dialysis machines. Since bacteria are considered to be too large to cross a dialyzer membrane, most dialysis providers use non-sterile water as a basis for the dialysate fluid. Lately, there is a growing body of scientific literature, pointing to the fact that toxins formed by excretions from bacteria may cross the dialyzer membrane. These toxins created by the bacteria may cross a membrane even though the bacteria themselves cannot cross it. These bacterial toxins may cause an inflammatory response in a dialysis patient. As a result, there is a push from the committees for dialysis standards that all the large dialysis machines use “ultra pure” (toxin and bacteria free dialysate) water and dialysate for each patient. Presently, the same dialysate is used for multiple patients. If these committees for dialysis standards succeed in such a requirement, the result would be an additional very large economic burden on dialysis clinics, insurance companies, and patients. Furthermore, all dialysate would be discarded after a single use in the dialyzer for each patient. Discarding dialysate after a single use would require about 90-120 liters of fresh (but not sterile) water, to be softened and purified usually by reverse osmosis in order to provide ultra pure dialysate for the next dialysis patient. This would be hugely expensive to the dialysis clinics involved.

The only existing system that regenerates and re-circulates dialysate is the REDY system that re-circulates about 6 liters of water for a total of 4 hours of dialysis treatment. The current REDY system does not utilize sterile water or ultra-pure dialysate. None of the sorbents or chemicals in the filter are considered to be sterile or free of bacteria and toxins.

Conversely, in various exemplary CRRT devices, less than one liter of fluid can be used in the dialysate circuit. In fact in some embodiments of the CRRT device, no more than 400 milliliters of fluid in the whole of the dialysate circuit are needed. As such, it is economically feasible to provide between 400 and about 1 liter or so of sterile ultra-pure water for dialysis, hemofiltration, or hemodiafiltration functions in an exemplary CRRT device that provides 24 hours of substantially continuous dialysis, hemofiltration, or hemodiafiltration for the patient. At present, an exemplary embodiment of the present CRRT device may have its sterile, ultra-pure dialysate exchanged every 24-96 hours. Furthermore, other portions of the dialysate regeneration system, which also contains some of the sterile, ultra-pure water and dialysate, as well as, chemicals, and filter cartridges with sorbents therein that are sterile and free of bacteria and toxins, may be changed every 12-96 hours.

Referring now to FIG. 14, the flow chart depicting a method process and/or protocol of installing and servicing an exemplary completely wearable CRRT device on a patient is provided. At step 800, a permanent catheter is installed in a patient. Existing dialysis devices are hooked to catheters that exit the patient's body at the neck or chest level. The catheter for an exemplary wearable CRRT device is longer than the prior used catheters and may exit the chest, but is long enough to reach the waist where the catheter provides an input and output (726, 727) for the input and output to the exemplary CRRT device. The catheter is tunneled under the skin to the level of the waist so that it could be hooked up to the exemplary CRRT device. Inside the patient's body, the catheter is implanted in the superior vena cava, the subclavian vein, the femoral vein, or the jugular vein of the patient. The catheter may be a double lumen catheter that extends from one of the vein connections down the patient's body, underneath the skin, to where it exits at or near the patient's waist for connection to an exemplary CRRT device.

After the catheter has been installed in the patient, the next step 802 is where the catheter input and output lumens are connected to the CRRT device (or wearable kidney) to be worn by the patient. The double lumen device is connected to the exemplary CRRT device under sterile conditions. Sterile conditions require that the nurse, doctor, or technician be scrubbed and is wearing a gown, mask, and perhaps sterile gloves. The patient is also wearing a sterile gown and cap and the connection of the catheter to the CRRT device is performed in a sterile room having an environment that is sterile much like a surgery room.

In prior situations where a patient goes to a dialysis clinic, the connection of the patient's internal catheter to the large dialysis machines in the clinic is not done under sterile conditions.

The sterile conditions are incorporated into the present exemplary method, protocol and procedure to exceed standards and aid in the mitigation of a risk of infection to the patient. An exemplary CRRT device worn by the patient for 24 hours, 7 day-a-week. As such, it is very important that whenever the blood circuit is open that a sterile environment and protocol be adhered to. Furthermore, all connections between the CRRT device and the patient's catheter are performed using sterile procedures that may include scrubbing such connections with antiseptic solutions. Such solutions may include alcohol, iodine, beta dine and other cleansing or sterilizing solutions.

At step 804, sterile and/or ultra pure dialysate is loaded into the dialysate circuit of the exemplary CRRT device. If the dialysate is not sterile and ultra pure then bacteria present in the non-sterile dialysate, though unable to cross the dialyzer membrane, may produce toxins that can cross the membrane, thereby causing inflammatory responses in the patient. As such, embodiments of the present CRRT device would use ultra pure dialysate that is free of toxins and would also use sterile water in the manufacture of such ultra pure dialysate. In some embodiments of the present invention, the level of sterility required for intravenous (IV) fluids would also be a requirement for the dialysate used in an exemplary embodiment. The USP has a requirement for sterile IV fluids. The USP also provides definitions for sterility and ultra pure.

Since embodiments with the present CRRT device require between 375 cc's and 1,000 cc's of dialysate as opposed to 90 to 120 liters of dialysate as required in the large existing dialysis machines, the use of ultra pure and sterile dialysate is economically feasible and also helps maintain the purity of the system and to mitigate risk of bacterial related infection or inflammation in the patient.

At step 806, it is understood that from time to time the dialyzer 730 may require replacement. In prior large dialysis machines, when the dialyzer requires replacement, a nurse or technician generally turns off the dialysis machine, puts on a pair of non-sterile gloves, clamps the tubing and changes the dialyzer. The nurse may not wear a mask, the patient may not be wearing a mask either, and the blood circuit incorporating the dialyzer is open to receive bacteria or infection that is floating within the non-sterile room.

At step 806, an exemplary dialyzer 730 is changed in a sterile environment similar to the sterile environment in which the patient's catheter was connection to the CRRT device. Again, the method, according to the present invention, requires that any time the blood circuit of an exemplary CRRT device is opened; it must be done in a sterile environment. The dialyzer may only be required to be changed every other day to once every fourteen to thirty days. An exemplary embodiment may require that the dialyzer be changed once a week. As such, a dialysis patient would be only be required to visit a dialysis clinic once a week, rather than every day or every other day, in order to have their dialyzer changed. A clinic would be required to have sterile room with an area for scrubbing where a nurse or technician can wash their hands, much like a surgeon prepares for an operation. The ends or tips of the dialyzer are cleaned and sterilized and then installed in the blood circuit by the nurse in a sterile environment.

It should be understood that in present day existing large CRRT machines, a single dialyzer is used for three days within the large CRRT machine and with as many patients as require dialysis over those three days. At the end of the third day, the dialyzer is changed because that's the way it is labeled by the manufacturer.

Embodiments of the present CRRT device may incorporate different sized dialyzers or more than one dialyzer depending on the needs or the prescription of the patient. For example, a young child may require a small dialyzer while an obese adult may require a larger or multiple dialyzers connected in series within an exemplary CRRT device. Such dialyzers may be required to be changed every two days or as infrequently as about every two weeks or longer.

As previously discussed, embodiments of the present CRRT device operate substantially 24 hours-a-day, 7 days-a-week while being worn by the dialysis patient. Since blood is continuously flowing through the blood circuit of the exemplary CRRT device, there is a concern that clotting may occur. As such, in step 808 heparin is continuously added near the beginning of the CRRT blood circuit. That is, heparin is added near where the input catheter that provides blood from the patient to the completely wearable CRRT device is connected. There is a reservoir 734 that holds a blood thinner or anticoagulant such as heparin with a dispensing mechanism such as a micro pump or other type of equivalent pump discussed herein that dispenses heparin continuously while the CRRT device is operating. Again, the CRRT device is designed to operate substantially continuously for 24 hours, 7 day-a-week. As such, heparin or other anticoagulation substance(s) would be continuously provided to the front end of the blood circuit, or at least, prior to the dialyzer 730. The reservoir may be a plastic bag or other cartridge that contains heparin and that can be replaceable by the patient. A prescribing physician will set and adjust the amount of heparin provided to the blood circuit, manually or under electronic control, according to medical criteria. The heparin ideally will be provided, not as a drip, but rather a flow or constant infusion of heparin into the blood circuit. The heparin would help mitigate the chances of the formation of a clot in the blood pump and/or dialyzer of an exemplary CRRT device.

From time to time the dialysate purification system will need to be changed either in part or in total. At step 810 the dialysate purification system is changed, in part or in total, every 12 to 96 hours. The dialysate purification system includes: (a) cartridge or cartridges that are used to regenerate and clean the dialysate of impurities, as well as the sterile ultra pure dialysate and additional additives and chemicals that are added to the dialysate circuit to help regenerate the dialysate. Such cartridges are shown in FIG. 12 as either single element 769 or multiple cartridge elements 768, 766, 762 and 760. Additional additives may be added from reservoir or fluid cartridge or bag 772 via pump 774. It is noted in FIG. 12 that there can be multiple reservoirs 772 and pumps 774 providing various different additives to the dialysate circuit. As discussed earlier, the filter cartridges contain sorbents that purify the dialysate and remove toxins. Those toxins are captured in the sorbents and once they are captured they, over time, saturate the sorbent powder and the filters must be replaced. It is also understood that the filters may be replaced over different periods of time or all at the same time as one or multiple filter units. As such, the filter cartridges can be removed and new filter cartridges can be inserted and/or connected into the dialysate circuit.

Since this is the dialysate circuit, and not the blood circuitry that is being interacted with, the patient in a clean but non-sterile environment can perform the exchanging or replacement of the filter cartridges and/or dialysate. That is, the exchanging of the dialysate and filter cartridges does not necessarily require a sterile environment. There is no opening of the blood system and in the unlikely case that bacteria enters the dialysate liquid since it is changed every day or so, there is not much time for bacteria to multiply and create a significant amount of toxins that could cross the dialyzer membrane barrier into the patient's bloodstream.

Again, the changing of the dialysate purification system may occur, depending upon the prescription, to be between half a day to every four days.

The ultra pure dialysate may be provided to the patient in the form of a plastic bag or cartridge container for the patient's use with the completely wearable CRRT device. As such, unlike any other dialysis system, a patient would be able to change key components in the dialysate loop of his or her wearable CRRT device.

Moving to step 812, the CRRT device operates using a power source 716. The power source would need to be recharged or replaced from time to time. The power source may be a rechargeable battery pack that can be easily changed out or connected to a recharger for recharging when the patient is resting in a chair or sleeping. An exemplary CRRT device would utilize a battery or power source that would allow the CRRT device to operate continuously for a minimum of 12 hours and perhaps a maximum of 36 hours. Regardless, because of the necessity of the dialysis device, it would be important to recharge or replace the battery or power source every 12-24 hours. As battery technology improves, one would understand and expect that the time between power source recharging or changes would increase.

Exemplary embodiments of the CRRT device may also provide an alarm or power gauge so that the patient will know when the power supply 716 is running low and is in need of being recharged or changed.

With respect to step 814, there are additives in the exemplary wearable CRRT device that are used to recharge the dialysate. These additives need to be replenished or replaced from time to time. There may also be other additives that are added to the blood circuit, such as anti-coagulants, vitamins, hormones, or other drugs that may also need to be replenished. As such, from time to time, various additives would have to be added to an exemplary CRRT device. In certain embodiments of the present CRRT device, such additives would be provided in the form of a cartridge or small fluid containing bags that can be removably inserted and/or connected to the required location or locations within the CRRT device. It is important to understand that these additives can be added by the patient without necessitating a trip to a dialysis clinic. Embodiments of the present wearable CRRT device may require additives to be added or replenished every 24-96 hours, depending on the prescription of the patient.

By following the protocol and method of installing and servicing an exemplary, completely wearable, CRRT device, cost of providing dialysis to dialysis patients can be kept in check while mitigating the risks of using a continuously wearable CRRT device. Furthermore, the risks of being connected, disconnected, and reconnected to a present day large scale or other type of dialysis machine would also be mitigated by using various exemplary embodiments of the present completely wearable CRRT device.

Many variations and embodiments of the above-described invention and method are possible. Although only certain embodiments of the invention and method have been illustrated in the accompanying drawings and described in the foregoing Detailed Description, it will be understood that the invention is not limited to the embodiments disclosed, but is capable of additional rearrangements, modifications and substitutions without departing from the invention as set forth and defined by the following claims. Accordingly, it should be understood that the scope of the present invention encompasses all such arrangements and is solely limited by the claims as follows:

What is claimed is:

1. A method of installing and servicing a completely wearable CRRT device comprising:
installing a double catheter in a patient from at least one of the superior vena cava, the subclavian vein and the jugular vein, under a patient's skin, to an exit location near the patient's waist;
connecting said double catheter to a completely wearable CRRT device in a sterile environment;
incorporating less than 1 liter of sterile dialysate into said completely wearable CRRT;
changing a dialyzer in said completely wearable CRRT device, in a sterile environment, after a first predetermined time period;
continuously adding an anticoagulation substance, by said completely wearable CRRT device, at an input side of said completely wearable CRRT device while said completely wearable CRRT device is operating;
changing a dialysate purification system after a second predetermined time period;
recharging or replacing a portable power source in said completely wearable CRRT device after a third predetermined time period.
exchanging the sterile dialysate in the completely wearable CRRT device with new sterile dialysate after a fourth predetermined time period; and
replenishing one or more additives in said completely wearable CRRT device.

2. The method of installing and servicing a completely wearable CRRT device of claim 1, wherein said incorporating of less than 1 liter of sterile dialysate is performed by inserting a fluid cartridge into said completely wearable CRRT device.

3. The method of installing and servicing a completely wearable CRRT device of claim 1, wherein said changing of said dialysate purification system comprises changing at least one filter cartridge.

4. The method of installing and servicing a completely wearable CRRT device of claim 1, wherein said changing of said dialysate purification system is performed by said patient.

5. The method of installing and servicing a completely wearable CRRT device of claim 1, wherein said first predetermined time period is one to fourteen days.

6. The method of installing and servicing a completely wearable CRRT device of claim 1, wherein said second predetermined period is 12 to 96 hours.

7. The method of installing and servicing a completely wearable CRRT device of claim 1, wherein said third predetermined period is 12 to 24 hours.

8. The method of installing and servicing a completely wearable CRRT device of claim 1, wherein said fourth predetermined time period is 24 to 96 hours.

9. The method of installing and servicing a completely wearable CRRT device of claim 1, wherein said sterile dialysate is also ultra pure dialysate.

10. The method of installing and servicing a completely wearable CRRT device of claim 1, wherein the exchanging the sterile dialysate is performed by the patient.

11. The method of installing and servicing a completely wearable CRRT device of claim 1, wherein replenishing one or more additives is performed by the patient.

12. A method of servicing a completely wearable CRRT device comprising:
changing a dialyzer in said completely wearable CRRT device, in a sterile environment, after a first predetermined time period;
continuously adding an anticoagulation additive, by said completely wearable CRRT device, at an input side of said completely wearable CRRT device while said completely wearable CRRT device is operating;
changing a dialysate purification system after a second predetermined time period;
recharging or replacing a portable power source in said completely wearable CRRT device after a third predetermined time period.
exchanging the sterile dialysate in the completely wearable CRRT device with new sterile dialysate after a fourth predetermined time period; and
replenishing one or more additives in said completely wearable CRRT device.

13. The method of servicing a completely wearable CRRT device of claim 12, wherein said first predetermined time period is between about one to about thirty days.

14. The method of servicing a completely wearable CRRT device of claim 12, wherein said second predetermined period is greater than 12 hours.

15. The method of servicing a completely wearable CRRT device of claim 12, wherein said third predetermined period is greater than 12 hours.

16. The method of servicing a completely wearable CRRT device of claim 12, wherein said fourth predetermined time period is 24 to 96 hours.

17. The method of servicing a completely wearable CRRT device of claim 12, wherein said changing said dialysate purification system is performed by a patient wearing said completely wearable CRRT device.

18. The method of servicing a completely wearable CRRT device of claim 12, wherein said exchanging of said sterile dialysate is performed by said patient.

19. The method of servicing a completely wearable CRRT device of claim 12, wherein exchanging said sterile dialysate requires an exchange of less than 1 liter sterile dialysate.

20. In a completely wearable CRRT device that operates with less than about 1 liter of dialysate, a method of servicing said completely wearable CRRT device comprising:
installing less than about 1 liter of sterile dialysate into said completely wearable CRRT device;
changing a dialyzer in said completely wearable CRRT device, every 2 to 30 days of device operation, in a substantially sterile environment;
changing a dialysate filter cartridge every 12 to 96 hours of device operation;
changing said less than about 1 liter of sterile dialysate, every 24 to 96 hours of device operation; and
adding dialysate additives to said completely wearable CRRT device every 24 to 96 hours of operation.
